(19) United States
(12) Plant Patent Application Publication
(10) Pub. No.: US 2004/0133952 P1
(43) Pub. Date: Jul. 8, 2004

(54) SHRUB ROSE PLANT NAMED 'RADCON'

(76) Inventor: Israel Montesino, Wilmington, DE (US)

Correspondence Address: BURNS DOANE SWECKER & MATHIS L L P, POST OFFICE BOX 1404, ALEXANDRIA, VA, 22313-1404, US

(21) Appl. No.: 10/335,865

(22) Filed: Jan. 3, 2003

(51) Int. Cl. A01H 5/00

(52) U.S. Cl. PLT/107

(57) ABSTRACT

A new and distinct variety of landscape shrub rose plant is provided which forms in abundance on a substantially continuous basis attractive single blossoms that are pink in coloration. The new variety is a spontaneous mutation of unknown causation of the ‘Radrazz’ variety (U.S. Plant Pat. No. 11,836) that forms vivid red single blossoms. The vegetation is vigorous and the growth habit is compact and mounding. Attractive ornamental satiny green foliage is formed. Excellent disease resistance to blackspot is exhibited. The new variety is particularly well suited for growing as distinctive ornamentation in the landscape.

### BOTANICAL/COMMERCIAL CLASSIFICATION

Rosa hybrida/Shrub Rose Plant

### VARIETAL DENOMINATION

cv. ‘Radcon’

### SUMMARY OF THE INVENTION

The new variety of Rosa hybrida landscape shrub rose plant of the present invention was discovered during March, 2001 while growing among a block of rose plants of the “Radrazz’ variety (U.S. Plant Pat. No. 11,836) growing in a nursery setting at West Grove, Pa., U.S.A. I was attracted to the new variety primarily because of its distinctive pink blossom coloration that was unlike that of the parental variety. The new variety is believed to be a spontaneous naturally-occurring mutation of the ‘Radrazz’ variety of unknown causation. Had I not discovered and preserved the original plant of the new variety it would have been lost to mankind.

It was found that the new variety of landscape shrub rose plant of the present invention possesses the following combination of characteristics:

(a) abundantly and substantially continuously forms attractive single blossoms that are pink in coloration unlike the vivid red blossoms of the ‘Radrazz’ variety (U.S. Plant Pat. No. 11,836),

(b) exhibits a compact and mounding growth habit,

(c) forms vigorous vegetation,

(d) forms attractive ornamental green foliage with a satiny finish, and

(e) exhibits excellent resistance to blackspot.

The new rose variety has attractive pink blooms, combined with a propensity for everblooming and blackspot resistance. The blooming cycle corresponds closely to that of the ‘Radrazz’ parental variety.

The new variety of the present invention can be readily distinguished from the parental ‘Radrazz’ variety through an inspection of the blossoms. More specifically, the new variety displays single blossoms that are pink in coloration rather than vivid red as are displayed by the ‘Radrazz’ variety.

The new variety well meets the needs of the horticultural industry. It can be grown to advantage as attractive ornamentation in parks, gardens, public areas, and residential landscapes. Accordingly, it is particularly well suited for growing throughout the landscape. The pink blossoms contrast nicely with the satiny green foliage.

The characteristics of the new variety have been found at West Grove, Pa., U.S.A., and at Wasco, Calif., U.S.A., to be homogeneous and stable and are strictly transmissible by asexual propagation such as budding, grafting, and the use of cuttings from one generation to another.

The new variety has been named the ‘Radcon’ variety.

### BRIEF DESCRIPTION OF THE PHOTOGRAPH

The accompanying photograph shows, as nearly true as it is reasonably possible to make the same in a color illustration of this character, a typical specimen of the flower of the new variety as well as a typical specimen of the flower of the parental ‘Radrazz’ variety. The rose plant from which the flowers were obtained were approximately two years of age and were grown in the ground outdoors on their own roots during September at West Grove, Pa., U.S.A.

FIG. 1 illustrates specimens of open flowers — plan view — obverse — with the flower of the new ‘Radcon’ variety being displayed on the right and the flower of the ‘Radrazz’ variety being displayed on the left. Dimensions in centimeters are included at the bottom of the photograph.

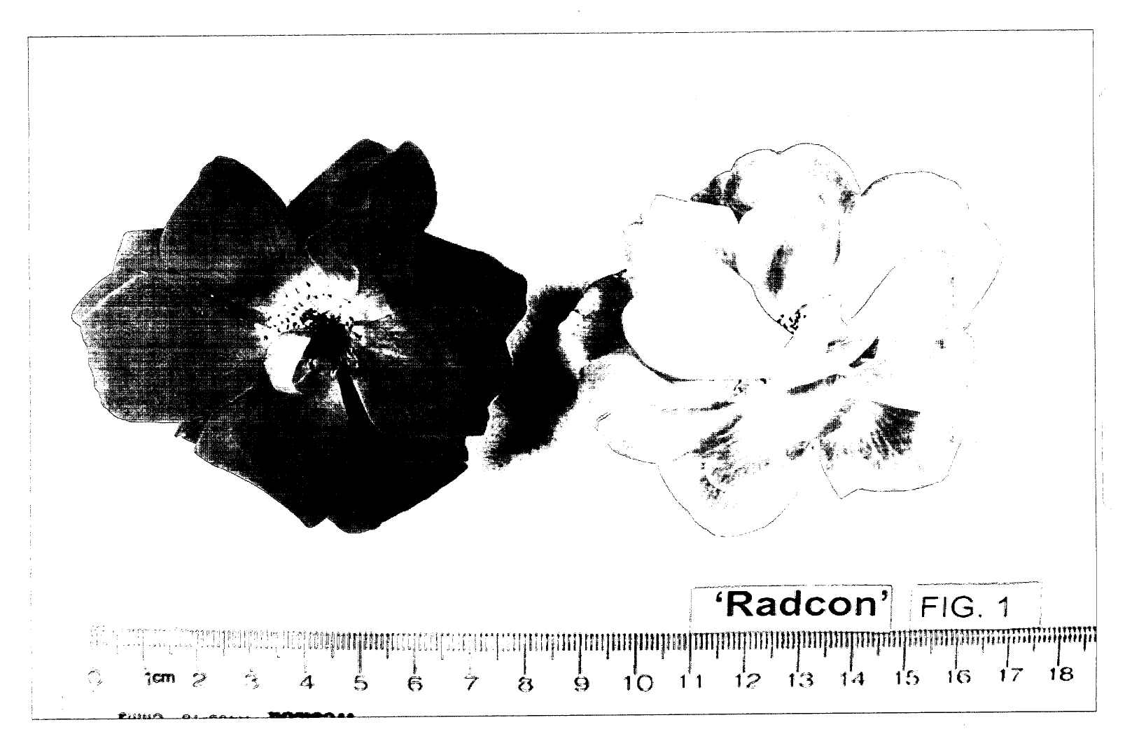

### DETAILED DESCRIPTION

The chart used in the identification of colors is that of The Royal Horticultural Society (R.H.S. Colour Chart). The description is based on approximately two year-old specimens of the new variety during September while grown in the ground outdoors on their own roots at West Grove, Pa., U.S.A.

Class: Landscape Shrub Rose.

Plant:

Height.—approximately 50 to 75 cm on average at the end of the growing season.

Width.—approximately 75 to 100 cm on average at the end of the growing season.

Habit.—compact, round, and bushy.

Branches:

Color.—young stems: near Yellow-Green Group 144C.

Leaves:

Leaflets.—number: 3, 5, and 7. — shape: ovate with a serrulate margin, rounded base, and acuminate tip. — size: approximately 7 cm in length on average. — serration: fine. — texture: smooth. — overall appearance: very dense, leathery, and medium green in coloration, with a light satiny finish on the ventral surface. — color (adult foliage): upper surface: Green Group 136B to Yellow-Green Group 147B.

Inflorescence:

Number of flowers.—commonly approximately 3 to 4 blooms on average in a cluster.

Flower.—form: single and informal. — diameter: approximately 6 cm. on average. — color (when blooming): upper surface: near Red Group 48D. — fragrance: faint. — petal form: wedge-shaped with a curled apex. — petal number: approximately 12 on average. — lasting quality: commonly approximately two weeks on the plant. — petal drop: very good with the petals commonly dropping cleanly and freely.

Development:

Vegetation.—vigorous and strong.

Blossoming.—abundant and substantially continuous.

Resistance to diseases.—excellent with respect to rose blackspot.

Propensity to form hips.—none observed to date.

I claim:

1. A new and distinct variety of landscape shrub rose plant characterized by the following combination of characteristics:
(a) abundantly and substantially continuously forms attractive single blossoms that are pink in coloration unlike the vivid red blossoms of the “Radrazz’ variety (U.S. Plant Pat. No. 11,836),
(b) exhibits a compact and mounding growth habit,
(c) forms vigorous vegetation,
(d) forms attractive ornamental green foliage with a satiny finish, and
(e) exhibits excellent resistance to blackspot; substantially as herein shown and described.
